(19) United States
(12) Patent Application Publication
(10) Pub. No.: US 2013/0012983 A1
(43) Pub. Date: Jan. 10, 2013

(54) Surgical Instrument with Flexible Shaft

(75) Inventor: Gennedy Kleyman, (US)

(73) Assignee: Tyco Healthcare Group LP, Mansfield, MA (US)

(21) Appl. No.: 13/543,918

(22) Filed: Jul. 9, 2012

Related U.S. Application Data
(60) Provisional application No. 61/505,600, filed on Jul. 8, 2011.

(51) Int. Cl. A61B 17/00; A61B 17/29; A61B 17/068

(52) U.S. Cl. 606/205; 606/1; 227/175.1

(57) ABSTRACT

A surgical instrument includes an elongate body, an elongate shaft, a collar, and an end effector. In particular, the elongate body defines a longitudinal axis, a lumen therethrough and an aperture in communication with the lumen. The elongate shaft is disposed at least partially within the lumen of the elongate body. The collar is mounted at least partially on the elongate shaft. The effector is detachably coupled with the elongate shaft. The elongate shaft is transitionable between a first position in which the elongate shaft is aligned with the longitudinal axis and a second position in which a portion of the elongate shaft having the collar mounted thereon transversely bends and protrudes through an aperture, thereby causing the end effector to move along the longitudinal axis with respect to the elongate body.

### CROSS-REFERENCE TO RELATED APPLICATIONS

This application claims the benefit of and priority to U.S. Provisional Patent Application No. 61/505,600, filed Jul. 8, 2011, the entire disclosure of which is incorporated by reference herein.

### BACKGROUND

1. Technical Field

The present disclosure relates to a surgical instrument, and more particularly, to a surgical instrument having a flexible shaft transversely bendable to adjust longitudinal position of an end effector coupled thereto.

2. Background of Related Art

In laparoscopic and endoscopic surgical procedures, a small incision or puncture is made in a patient's body, e.g., in the abdomen, to provide an entry point for a surgical device. When compared to the larger incisions typically found in traditional procedures, both trauma to the patient and recovery time are reduced for procedures involving small incisions. Due to the relatively small interior dimensions of the access devices used in endoscopic procedures, only the elongated, small diametered instrumentation may be used to access the internal body cavities and organs.

During such procedures, surgical objects, such as surgical access devices, e.g., trocar and cannula assemblies, are inserted into the patient's body through the incision in tissue. In general, prior to the introduction of the surgical object into the patient's body, insufflation gases are used to enlarge the area surrounding the target surgical site to create a larger, more accessible work area.

The surgeon is then able to perform the procedure within the abdominal cavity by manipulating the instruments that have been extended through the access assemblies. The manipulation of such instruments within the internal body is similarly limited by both spatial constraints and the need to maintain the body cavity in an insufflated state.

Accordingly, there is a need for a surgical instrument that enables the user to manipulate the position of the end effector within the internal body limited by spatial constraints.

### SUMMARY

In accordance with an embodiment of the present disclosure, there is provided a surgical instrument including an elongate body, an elongate shaft, a collar, an end effector. In particular, the elongate body defines a longitudinal axis, a lumen therethrough, and an aperture in communication with the lumen. The elongate shaft is disposed at least partially within the lumen of the elongate body. The collar is mounted at least partially on the elongate shaft. The end effector is detachably coupled with the elongate shaft, wherein the elongate shaft is transitionable between a first position in which the elongate shaft is aligned with the longitudinal axis and a second position in which a portion of the elongate shaft having the collar mounted thereon transversely bends and protrudes through the aperture, thereby causing the end effector to move along the longitudinal axis with respect to the elongate body.

The elongate shaft may be a flexible member configured for transverse bending with respect to the longitudinal axis. The elongate body may define a plurality of circumferentially arranged apertures configured and dimensioned for passage therethrough of the portion of the elongate shaft having the collar mounted thereon. The elongate body may include an elastic sheath covering the aperture. The collar may be an O-ring. The collar may be disposed adjacent the aperture defined in the elongate body. The collar may have a smaller inner diameter than an outer diameter of the elongate shaft. The collar may be monolithically formed with the elongate shaft.

In an embodiment, the surgical instrument may further include a ratchet assembly including a plurality of teeth on an inner wall of the elongate body and corresponding teeth on an outer surface of the elongate shaft. In particular, the plurality of teeth on the inner wall of the elongate body and the corresponding teeth on the outer surface of the elongate shaft may be configured for proximal movement of the elongate shaft with respect to the elongate body. The plurality of teeth on the inner wall of the elongate body may diametrically oppose each other. The plurality of teeth on the outer surface of the elongate shaft may also diametrically oppose each other.

The elongate body may be rotatable with respect to the elongate shaft to engage the plurality of teeth disposed on the inner wall of the elongate body and the corresponding teeth on the outer surface of the elongate shaft. The plurality of teeth may be disposed adjacent a proximal end portion of the elongate body.

The surgical instrument may further include a handle assembly including a rotatable member, wherein rotation of the rotatable member imparts rotation to the elongate body.

In an embodiment, the end effector is a stapling device. Alternatively, the end effector may be a grasping assembly including a pair of jaws. The pair of jaws may be movable between an open position in which the jaws are spaced apart when the elongate shaft is in the first position and a closed position in which the jaws are disposed in a juxtaposed relation when the elongate shaft is in the second position.

### BRIEF DESCRIPTION OF THE DRAWINGS

Various embodiments of the present disclosure are described hereinbelow with reference to the drawings, wherein:

FIG. 1 is a perspective view of a surgical instrument in accordance with an embodiment of the present disclosure;

FIG. 2 is a top view of the surgical instrument of FIG. 1;

FIG. 3 is a side view of the surgical instrument of FIG. 1;

FIG. 4 is a partial perspective view of the distal region of the surgical instrument of FIG. 1;

FIG. 5 is a partial longitudinal cross-sectional view of the surgical instrument of FIG. 4 illustrating a flexible elongate shaft in a neutral state;

FIG. 6 is a partial longitudinal cross-sectional view of the surgical instrument of FIG. 4 illustrating a portion of the flexible elongate shaft transversely bent;

FIG. 7 is a cross-sectional view of an elongate body of the surgical instrument of FIG. 1 illustrating a pair of opposing openings defined in the elongate body;

FIG. 8 is a cross-sectional view of an elongate body for use with the surgical instrument of FIG. 1 illustrating a plurality of openings circumferentially defined in the elongate body;

FIG. 9 is a cross-sectional view of an elongate body for use with the surgical instrument of FIG. 1 illustrating an elongate body openings having elastic covers;

FIG. 10 is a cross-sectional view of an elongate body and a flexible shaft having a collar configured and dimensioned to facilitate passage through the openings;

FIG. 11 is a cross-sectional view of an elongate body and a flexible shaft having a collar configured and dimensioned to facilitate passage through the openings defined in multiple directions;

FIG. 12A is a partial longitudinal cross-sectional view of a ratchet assembly for use with the surgical instrument of FIG. 1 illustrating the ratchet assembly in a locked state;

FIG. 12B is a partial longitudinal cross-sectional view of the ratchet assembly of FIG. 12A in a disengaged state;

FIG. 13 is a cross-sectional view of the ratchet assembly of FIG. 12B illustrating engaging mechanism;

FIG. 14 is a perspective view of a surgical instrument in accordance with another embodiment of the present disclosure;

FIG. 15 is a partial longitudinal cross-sectional view of the surgical instrument of FIG. 14 illustrating a flexible shaft in a neutral state; and

FIG. 16 is a partial longitudinal cross-sectional view of the surgical instrument of FIG. 14 illustrating a portion of the flexible shaft transversely bent.

### DETAILED DESCRIPTION OF EMBODIMENTS

Embodiments of the present disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In the following description, well-known functions or constructions are not described in detail to avoid obscuring the present disclosure in unnecessary detail.

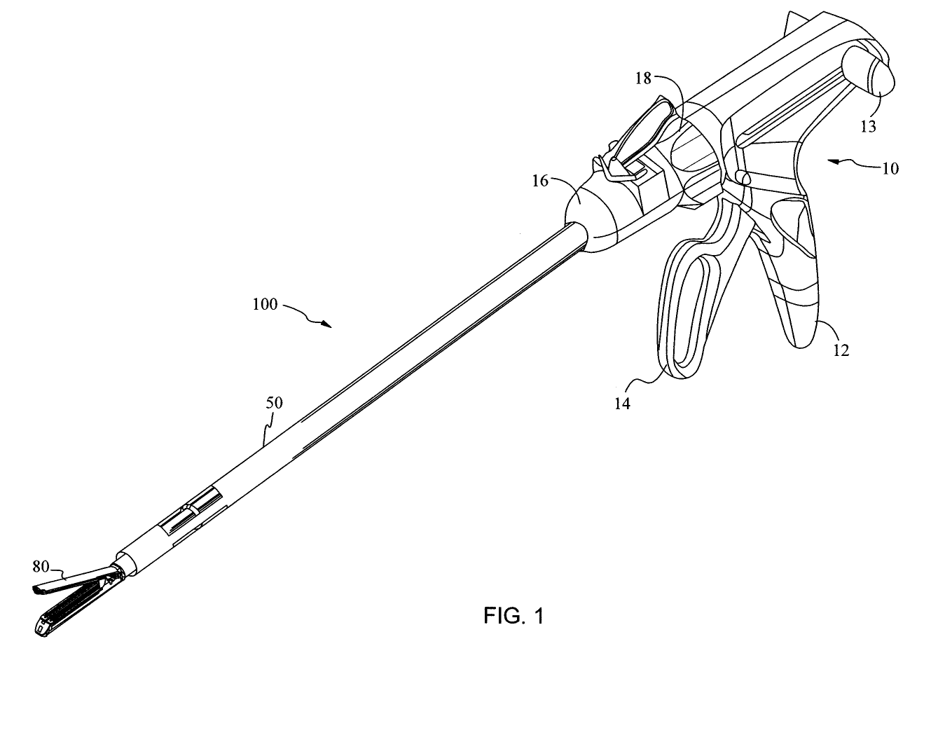

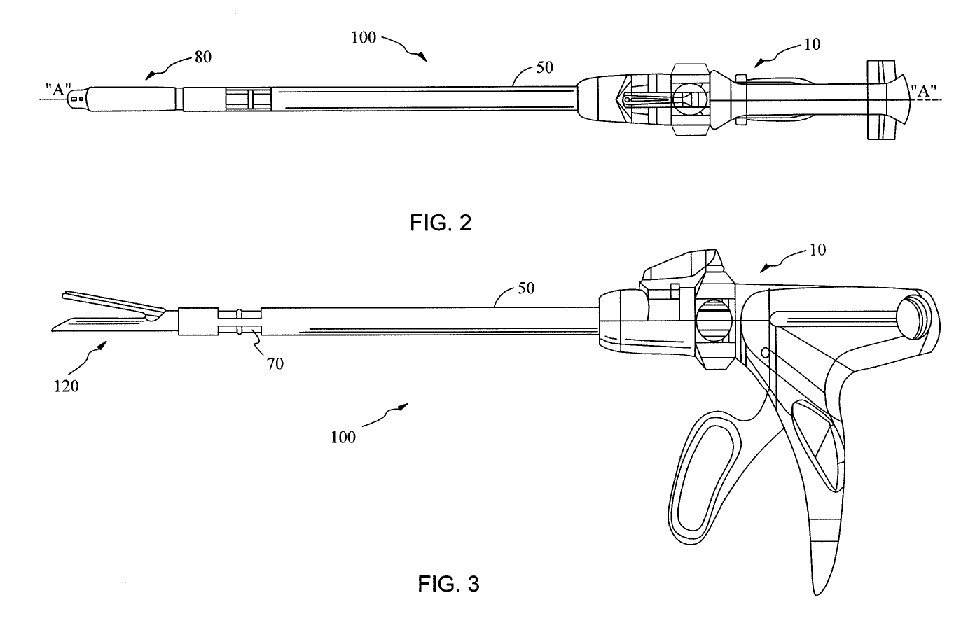

With reference to FIGS. 1-3, there is illustrated an endoscopic surgical instrument 100 in accordance with an embodiment of the present disclosure. In the interest of brevity, this disclosure will focus primarily on the structures that enable longitudinal movement of an end effector 80. A detailed discussion of the remaining components and method of use of surgical instrument 100 is disclosed in U.S. Pat. No. 6,953,139, the entire disclosure of which is incorporated herein by reference.

Surgical instrument 100 includes a handle assembly 10, an elongate body 50 extending from handle assembly 10 and an end effector 80. Surgical instrument 100 defines a longitudinal axis “A-A.” Handle assembly 10 includes a stationary handle member 12, a movable handle member 14, and a barrel portion 16. A rotatable member 18 is mounted on the distal end of barrel portion 16 to facilitate rotation of elongated body 50 with respect to handle assembly 10. A pair of retraction knobs 13 is movably positioned along barrel portion 16 for manually repositioning an actuation shaft of stapling device 120 to a proximal direction.

Elongate body 50 is configured for use in closed procedures, e.g., laparoscopic, endoscopic, and arthroscopic procedures. However, elongate body 50 may be shortened or removed for use in open procedures. End effector 80 is detachably coupled to handle assembly 10 via a flexible elongate shaft 70 extending through elongate body 50. Flexible elongate shaft 70 is of a highly flexible and pliable nature and may include thin control wires (not shown) to manipulate flexible elongate shaft 70, as will be discussed below.

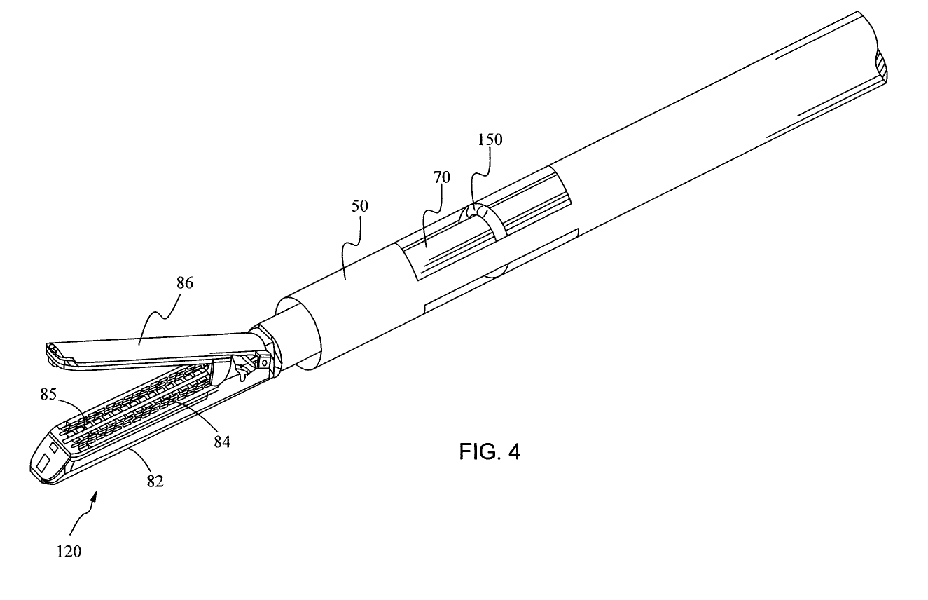

With reference now to FIG. 4, end effector 80 is a stapling device 120. Endoscopic surgical instrument 100 may be configured and adapted for use with end effectors having alternative configurations such as, for example, clip applier, vessel sealing devices, circular stapling devices, dissectors, retractors, cutters, graspers.

With reference now to FIGS. 4-6, stapling device 120 is releasably secured to a distal end portion of flexible elongate shaft 70. In particular, stapling device 120 includes a cartridge assembly 82 housing a plurality of surgical staples and an anvil assembly 86 movably secured in relation to cartridge assembly 82. Anvil assembly 86 is movable from an open position in which anvil assembly 86 is spaced apart from cartridge assembly 82 to a closed position in which anvil assembly 86 is substantially adjacent cartridge assembly 82. In particular, cartridge assembly 82 defines rows of staple containing slots 84 and a knife slot 85 defined between rows of staple slots 84. Stapling device 120 is configured to apply at least one row of staples. However, stapling device 120 may have various staple line lengths and configurations. Anvil assembly 86 includes a plurality of staple deforming concavities 88.

With continued reference to FIGS. 4-6, flexible elongate shaft 70 is disposed within elongate body 50. Flexible elongate shaft 70 is coupled to handle assembly 10 and stapling device 120. In particular, flexible elongate shaft 70 includes a collar 150 at least partially surrounding flexible elongate shaft 70. Collar 150 is an O-ring. Collar 150 is made from an elastomer such as, e.g., polyurethane, polyethylene, silicone, and the like. Elongate body 50 defines a pair of opposing openings 54 configured and dimensioned to enable a portion of flexible elongate shaft 70 containing collar 150 to transversely bend and protrude through opening 54.

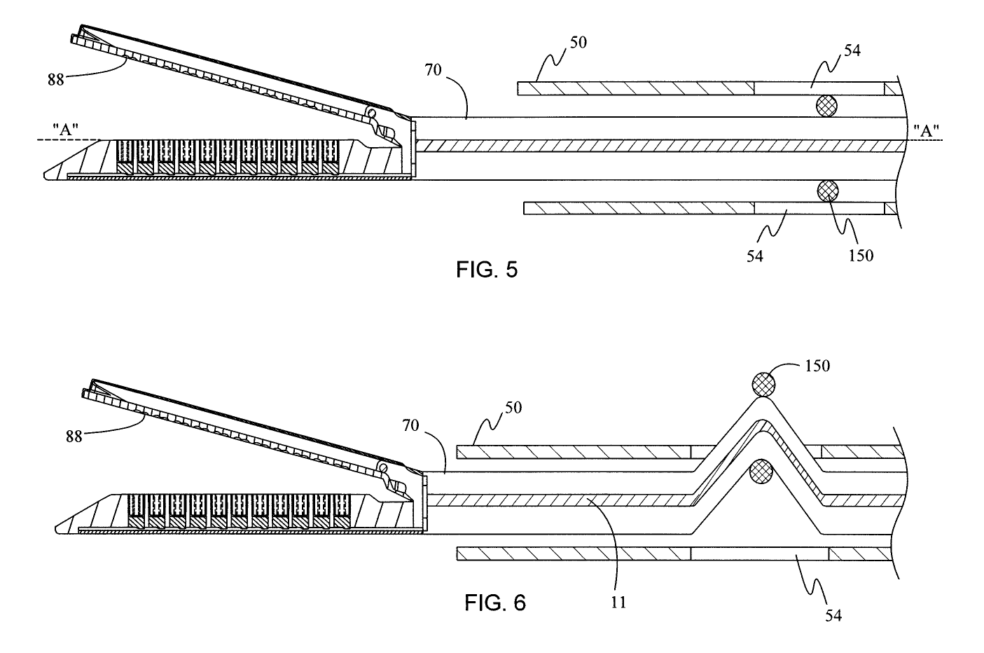

With continued reference to FIGS. 5 and 6, flexible elongate shaft 70 is transitionable between a neutral state in which flexible elongate shaft 70 is aligned with longitudinal axis “A-A” (FIG. 5) and a deformed/transitioned state in which a portion of flexible elongate shaft 70 containing collar 150 is at least partially offset with respect to longitudinal axis “A-A” (FIG. 6). Specifically, in the deformed/transitioned state, the portion of flexible elongate shaft 70 containing collar 150 at least partially protrudes through opening 54 defined in elongate body 50. In this manner, by transitioning flexible elongate shaft 70 between the neutral state and the deformed/transitioned state, stapling device 120 may be moved along longitudinal axis “A-A” to a desired longitudinal position.

With continued reference to FIGS. 5 and 6, flexible elongate shaft 70 is concentrically arranged within elongate body 50. An actuation rod 11 is disposed within flexible shaft 70 and is operatively coupled with handle assembly 10 to actuate firing of staples. In the interest of brevity, and as not to obscure the present disclosure, the structural and functional features of actuation rod 11 will not be discussed herein.

Collar 150 is mounted on flexible elongate shaft 70 adjacent openings defined in elongate body 50. In particular, collar 150 has a smaller outer diameter than that of elongate body 50, such that when flexible elongate shaft 70 is in the neutral state, collar 150 disposed around at least a portion of flexible elongate shaft 70 does not protrude through opening 54 defined in elongate body 50. In addition, collar 150 has an inner diameter smaller than that of flexible elongate shaft 70 such that collar 150 applies compressive force against flexible elongate shaft 70 to facilitate bending of a portion of flexible elongate shaft 70 adjacent collar 150.

With particular reference now to FIG. 6, collar 150 and the portion of flexible elongate shaft 70 containing collar 150 are configured and dimensioned for passage through opening 54. For example, a pair of control wires (not shown) may be utilized to effect bending of flexible elongate shaft 70 from a remote location, such as, for example, handle assembly 10. In addition, it is contemplated that the bending mechanism may be electrically or gas powered.

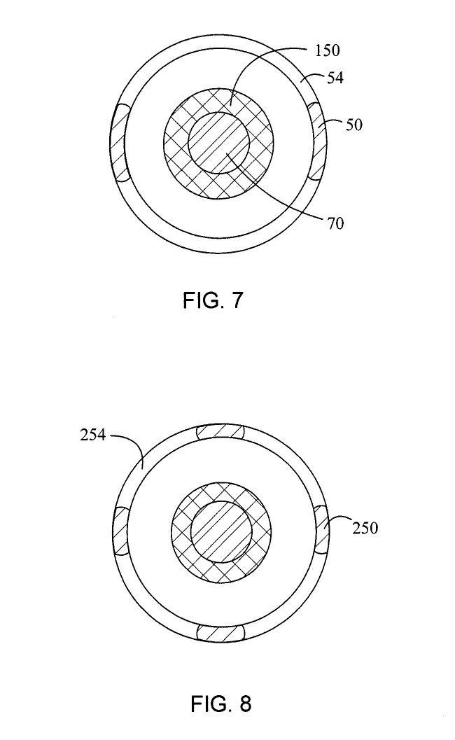

With reference to FIGS. 1 and 7, elongate body 50 of surgical instrument 100 defines a pair of diametrically opposing openings 54 to accommodate passage of collar 150 and the portion of flexible elongate shaft 70 containing collar 150 therethrough. However, it is also envisioned that an elongate body 250 may define a plurality of circumferentially defined openings 254 in multiple directions, as shown in FIG. 8. Such configuration enables transverse bending of flexible elongate shaft 70 in multiple directions. In this manner, by allowing bending of flexible elongate shaft 70 in multiple directions, manipulation of stapling device 120 along the longitudinal axis “A-A” is not limited by the orientation of bi-directional openings 54 defined in elongate body 50, as shown in FIG. 7.

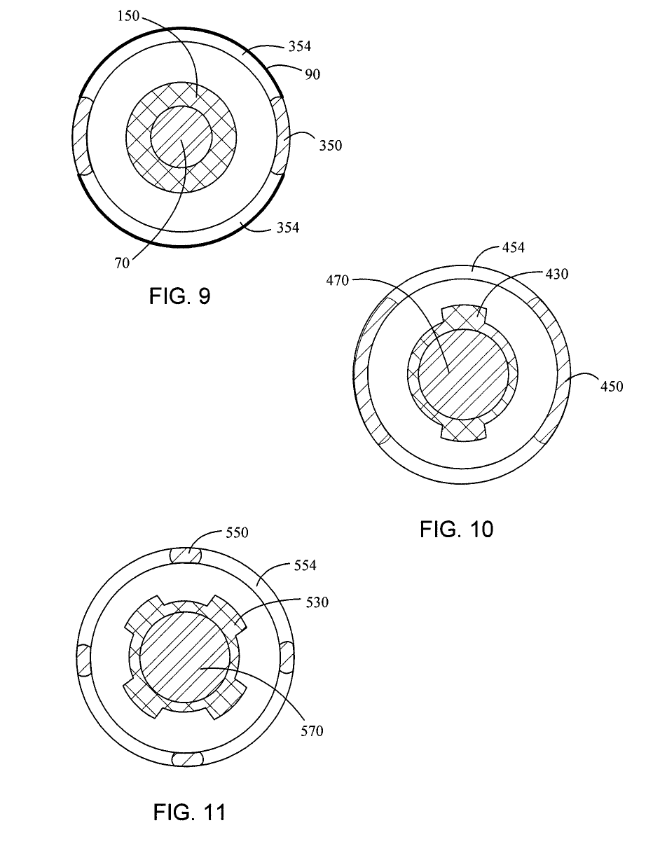

With reference to FIG. 9, it is further contemplated that an opening 354 defined in an elongate body 350 may include a sheath/cover 90 having biocompatible and elastic features. Sheath 90 is affixed to elongate body 350 and covers openings 354. The elastic characteristic of sheath 90 enables sheath 90 to expand and accommodate protrusion of flexible elongate shaft 70 and collar 150. Under such configuration, the risk of body organs or tissue being trapped or pinched in opening 354 is minimized.

With reference now to FIG. 10, it is also contemplated that a collar 430 may have a non-uniform annular cross-section. Specifically, collar 430 includes protruding portions that are configured and dimensioned for passage through opening 454 defined in elongate body 450. In this manner, the size of opening 454 can be reduced to further reduce possible pinching or trapping of organs or tissue in opening 454. In addition, the diameter of overall elongate body 450 may be reduced and/or the diameter of flexible elongate shaft 470 may be increased while retaining the same dimensions of elongate body 50. The reduced diameter of elongate body 450 may be beneficial in a minimally invasive surgery by requiring a smaller opening or incision in the patient and the larger diameter of flexible elongate shaft 470 may provide the desired rigidity or the structural integrity of flexible elongate shaft 470. In order to further facilitate transverse bending of flexible elongate shaft 570 in multiple directions, elongate body 550 may define a plurality of openings 554 in multiple directions, as shown in FIG. 11.

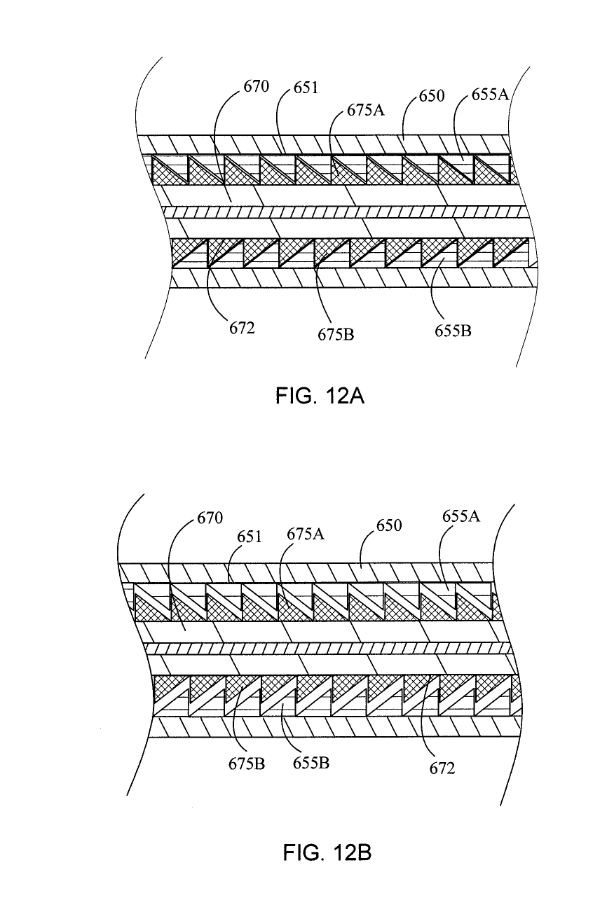

With reference now to FIGS. 12A and 12B, it is further envisioned that surgical instrument 100 may include a ratchet assembly. An elongate body 650 includes a plurality of teeth 655A, 655B on an inner wall 651 thereof. In addition, flexible elongate shaft 670 disposed within elongate body 650 also includes a plurality of corresponding teeth 675A, 675B on an outer surface 672 thereof. Teeth 655A, 655B disposed on inner wall 651 of elongate body 650 engage teeth 675A, 675B disposed on outer surface 672 of elongate shaft 670. In particular, the plurality of teeth 655A, 655B, 675A, 675B are such that that flexible elongate shaft 670 is movable in a single direction relative to elongate body 650 when teeth 655A, 655B, 675A, 675B are engaged (FIG. 12A). In particular, teeth 655A, 655B, 675A, 675B are configured to enable proximal movement of flexible elongate shaft 670 relative to elongate body 650, but teeth 655A, 655B, 675A, 675B inhibit relative distal movement of flexible elongate shaft 670.

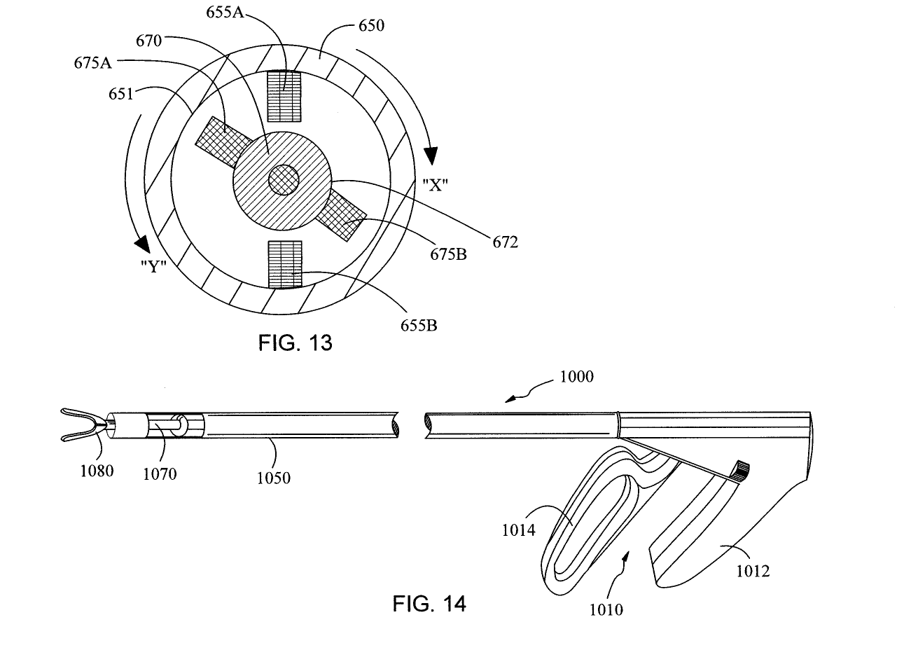

With reference to FIG. 13, teeth 655A, 655B disposed on inner wall 651 of elongate body 650 diametrically oppose each other. Similarly, teeth 675A, 675B on outer surface 672 of flexible elongate shaft 670 also diametrically oppose each other. In this manner, flexible elongate shaft 670 locked in a deformed/transition state by teeth 655A, 655B, 675A, 675B may be released from the locked state by rotating rotatable member 18 on handle assembly 10 which imparts rotation to elongate body 650. Specifically, rotation of elongate body 650 in either direction “X,” “Y” disengages teeth 655A, 655B, 675A, 675B. It is envisioned that in order to minimized interference with transverse bending of flexible elongate shaft 670 through opening defined in elongate body 670, teeth 655A, 655B, 675A, 675B are disposed proximal of the openings, for example, adjacent handle assembly 10.

In use, an incision is made through the abdominal wall of a patient and an access cannula (not shown) is inserted therethrough. The access cannula is connected to a source of insufflation fluid for creating an operative space within a body cavity. Surgical instrument 100 is inserted through the access cannula and into the surgical site within the body cavity. Through the use of, for example, the control wires, flexible elongate shaft 70 may be longitudinally moved to position end effector 80 in a desired surgical site. Thereafter, the surgeon may perform the desired surgical procedure. In the case of surgical instrument 100, the surgeon may actuate the handle assembly 10 to effect surgical stapling of tissue.

In accordance with another embodiment of the present disclosure, a surgical grasping instrument 1000 is illustrated in FIGS. 14-16. Grasping instrument 1000 includes a handle assembly 1010, a grasping assembly 1080 and an elongate member 1050 extending distally from handle assembly 1010 and terminating in grasping assembly 1080. Handle assembly 1010 includes a stationary handle member 1012 and a movable handle member 1014. Squeezing movable handle member 1014 toward stationary handle member 1012 actuates grasping assembly 1080.

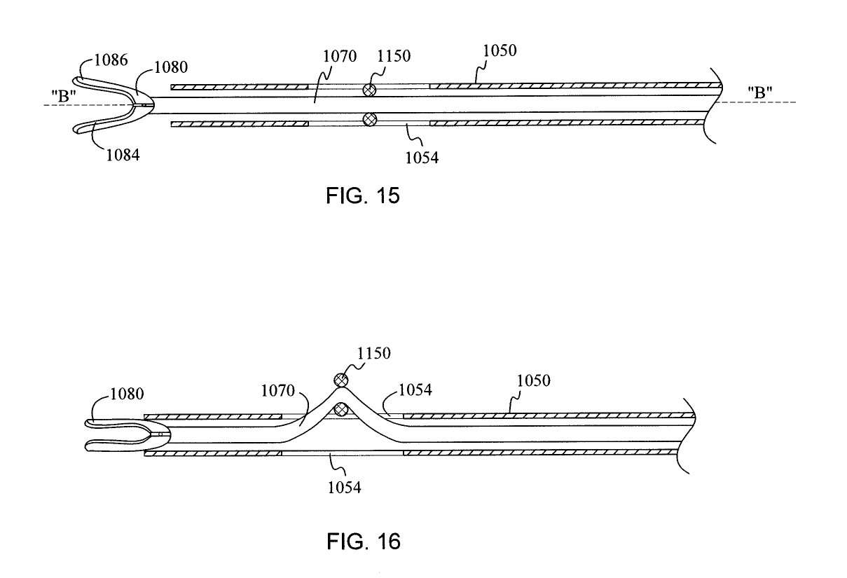

Elongate body 1050 is configured for use in closed procedures, e.g., laparoscopic, endoscopic, and arthroscopic procedures. However, elongate body 1050 may be shortened or removed for use in open procedures. Grasping assembly 1080 is detachably coupled to handle assembly 1010 via a flexible elongate shaft 1070 extending through elongate body 1050. Flexible elongate shaft 1070 is of a highly flexible and pliable nature to enable transverse bending with respect to the longitudinal axis defined by longitudinal axis “B-B” (FIG. 15).

With continued reference now to FIGS. 15 and 16, grasping assembly 1080 is releasably secured to a distal end portion of flexible elongate shaft 1070. In particular, grasping assembly 1080 includes a pair of jaws 1084, 1086. The pair of jaws 1084, 1086 is movable between an open position in which jaws 1084, 1086 are spaced apart and a closed position in which jaws 1084, 1086 are disposed in a juxtaposed relation to clamp, e.g., tissue or other surgical instruments, therebetween.

Flexible elongate shaft 1070 is disposed within elongate body 1050. Flexible elongate shaft 1070 is coupled to handle assembly 1010 and grasping assembly 1080. In particular, flexible elongate shaft 1070 includes a collar 1150 at least partially surrounding flexible elongate shaft 1070. Collar 1150 is an O-ring. Elongate body 1050 defines a pair of diametrically opposing openings 1054 configured and dimensioned to enable a portion of flexible elongate shaft 1070 containing collar 1150 to protrude through opening 1054.

With continued reference to FIGS. 15 and 16, flexible elongate shaft 1070 is transitionable between a neutral state in which flexible elongate shaft 1070 is aligned with longitudinal axis “B-B” and a deformed/transitioned state in which a portion of flexible elongate shaft 1070 containing collar 1150 is at least partially offset with respect to longitudinal axis “B-B.” Specifically, in the deformed/transitioned state, collar 1150 along with the portion of flexible elongate shaft 1070 containing collar 1150 at least partially protrudes through opening 1054 defined in elongate body 1050. In this manner, by transitioning flexible elongate shaft 1070 between the neutral state and the deformed/transitioned state, grasping assembly 1080 may be moved along longitudinal axis “B-B” to a desired longitudinal position.

Flexible elongate shaft 1070 is concentrically arranged within elongate body 1050. Flexible shaft 1070 is operatively coupled with handle assembly 1010. Collar 1150 is mounted on flexible elongate shaft 1070 adjacent opening 1054 defined in elongate body 1050. In particular, collar 1150 has a smaller outer diameter than that of elongate body 1050, such that when flexible elongate shaft 1070 is in the neutral state, collar 1150 mounted on flexible elongate shaft 1070 remains within elongate body 1050. In addition, collar 1150 has an inner diameter smaller than that of flexible elongate shaft 1070 such that collar 1150 applies compressive force against flexible elongate shaft 1070 to facilitate transverse bending of a portion of flexible elongate shaft 1070 adjacent collar 1150.

With particular reference now to FIG. 16, in the deformed/transitioned state, collar 1150 and the portion of flexible elongate shaft 1070 containing collar 1150 protrude through opening 1054. For example, a pair of control wires (not shown) may be utilized to effect transverse bending of flexible elongate shaft 1070, whereby squeezing of movable handle member 1014 toward stationary handle member 1012 moves grasping assembly 1080 in a proximal direction and collar 1150 protrudes at least partially through opening 1054.

When handle assembly 1010 is actuated, grasping assembly 1080 is moved proximally and collar 1150 at least partially protrudes out of one of the pair of diametrically opposing openings 1054. Specifically, as grasping assembly 1080 moves proximally the pair of jaws 1084, 1086 is at least partially received within elongate body 1050, whereby grasping assembly 1080 is transitioned into the closed state.

It is further contemplated that grasping instrument 1000 may include a ratchet mechanism to enable relative movement of grasping assembly 1080 in a single direction, such as proximal direction and inhibit movement in the distal direction. Under such configuration, tissue or a surgical instrument clamped between jaws 1084, 1086 may remain clamped therebetween during the surgical procedure. As discussed hereinabove, it is also envisioned that elongate body 1050 may define a plurality of openings to enable transverse bending of flexible elongate shaft 1070 in multiple directions. In addition, collar 1150 may have a non-uniform annular cross- section to facilitate passage through openings 1054 defined in elongate body 1050. Furthermore, opening 1054 defined in elongate body 1050 may include a sheath affixed thereto to inhibit, e.g., pinching or trapping of tissue, within opening 1054.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A surgical instrument comprising:
an elongate body defining a longitudinal axis and a lumen therethrough, the elongate body defining an aperture in communication with the lumen;
an elongate shaft disposed at least partially within the lumen of the elongate body;
a collar mounted at least partially on the elongate shaft; and
an end effector coupled with the elongate shaft, wherein the elongate shaft is transitionable between a first position in which the elongate shaft is aligned with the longitudinal axis and a second position in which a portion of the elongate shaft having the collar mounted thereon transversely bends and protrudes through the aperture, thereby causing the end effector to move along the longitudinal axis with respect to the elongate body.

2. The surgical instrument according to claim 1, wherein the elongate shaft is a flexible member configured for bending transversely with respect to the longitudinal axis.

3. The surgical instrument according to claim 1, wherein the elongate body defines a plurality of circumferentially arranged apertures configured and dimensioned for passage therethrough the collar and the portion of the elongate shaft having the collar mounted thereon.

4. The surgical instrument according to claim 1, wherein the elongate body includes an elastic sheath covering the aperture.

5. The surgical instrument according to claim 1, wherein the collar is an O-ring.

6. The surgical instrument according to claim 1, wherein the collar is disposed adjacent the aperture defined in the elongate body.

7. The surgical instrument according to claim 1, wherein the collar has a smaller inner diameter than an outer diameter of the elongate shaft.

8. The surgical instrument according to claim 1, wherein the collar is monolithically formed with the elongate shaft.

9. The surgical instrument according to claim 1, further comprising a ratchet assembly including a plurality of teeth on an inner wall of the elongate body and corresponding teeth on an outer surface of the elongate shaft.

10. The surgical instrument according to claim 9, wherein the plurality of teeth on the inner wall of the elongate body and the corresponding teeth on the outer surface of the elongate shaft are configured for proximal movement of the elongate shaft with respect to the elongate body.

11. The surgical instrument according to claim 9, wherein the plurality of teeth on the inner wall of the elongate body diametrically oppose each other.

12. The surgical instrument according to claim 9, wherein the plurality of teeth on the outer surface of the elongate shaft diametrically oppose each other.

13. The surgical instrument according to claim 9, wherein the elongate body is rotatable with respect to the elongate shaft to engage the plurality of teeth disposed on the inner wall of the elongate body and the corresponding teeth on the outer surface of the elongate shaft.

14. The surgical instrument according to claim 10, wherein the plurality of teeth are disposed adjacent a proximal end portion of the elongate body.

15. The surgical instrument according to claim 10, further comprising a handle assembly including a rotatable member, wherein rotation of the rotatable member imparts rotation to the elongate body.

16. The surgical instrument according to claim 1, wherein the end effector is a stapling device.

17. The surgical instrument according to claim 1, wherein the end effector is a grasping assembly including a pair of jaws.

18. The surgical instrument according to claim 14, wherein the pair of jaws is movable between an open position in which the jaws are spaced apart when the elongate shaft is in the first position and a closed position in which the jaws are disposed in a juxtaposed relation when the elongate shaft is in the second position.

19. The surgical instrument according to claim 1, wherein the end effector is detachably coupled with the elongate shaft.
